(19) United States
(12) Patent Application Publication
(10) Pub. No.: US 2015/0376511 A1
(43) Pub. Date: Dec. 31, 2015

(54) PRODUCTION OF JET FUEL-RANGE HYDROCARBONS FROM CELLULOSIC BIOMASS

(71) Applicant: PHILLIPS 66 COMPANY, Houston, TX (US)

(72) Inventors: Edgar LOTERO, Cleveland, OK (US); Sourabh S. PANSARE, Bartlesville, OK (US); Kristi A. Fjare, Ponca City, OK (US)

(73) Assignee: PHILLIPS 66 COMPANY, Houston, TX (US)

(21) Appl. No.: 14/744,945

(22) Filed: Jun. 19, 2015

Related U.S. Application Data
(60) Provisional application No. 62/017,384, filed on Jun. 26, 2014.

(51) Int. Cl. C10G 3/00; C07C 45/72; C07D 307/12; C07C 45/45; C10L 1/04; C07C 29/09

(52) U.S. Cl. CPC C10G 3/50; C10G 3/42; C10L 1/04; C07C 29/09; C07D 307/12; C07C 45/45; C07C 45/72; C10L 2200/0469; C10L 2270/04

(57) ABSTRACT

The present invention is related to the production of jet fuel-range hydrocarbons from cellulose/hemicellulose derived alcohols, such as pentanediol and/or hydroxymethyl-tetrahydrofuran which is also known as tetrahydrofurfuryl alcohol. The alcohols are spliced or dimerized through Guerbet chemistry where longer chain organic molecules are formed using an alcohol condensation catalyst. The organic molecules are hydrotreated to remove oxygen to yield jet fuel-range hydrocarbons.

### CROSS-REFERENCE TO RELATED APPLICATIONS

This application is a non-provisional application which claims the benefit of and priority to U.S. Provisional Application Ser. No. 62/017,384 filed Jun. 26, 2014, titled “Production of Jet Fuel-Range Hydrocarbons from Cellulosic Biomass,” which is hereby incorporated by reference in its entirety.

### STATEMENT REGARDING FEDERALLY SPONSORED RESEARCH OR DEVELOPMENT

None.

### FIELD OF THE INVENTION

This invention relates to producing hydrocarbon fuel from biomass and more particularly to producing jet fuel range hydrocarbon fuel from biomass.

### BACKGROUND OF THE INVENTION

It is highly desirable to find an economic process for converting biomass to fuel. Many inefficient processes are known that convert biomass to hydrocarbon fuels, but the processes are not very productive and are not currently cost competitive with petroleum sourced hydrocarbon fuels. Cellulose, hemicellulose, and lignin are the three main constituents of biomass. When the cellulose and hemicellulose portions of biomass are subjected to acid hydrolysis, the sugar polymers get converted to sugar monomers. These monomers, on subsequent dehydration, form furfural and hydroxymethyl furfural. Hydrogenolysis of furfural and hydroxymethyl furfural results in the formation of a mixture of 1,4-pentanediol, hydroxymethyl tetrahydrofuran, and methyl tetrahydrofuran. It is possible to obtain pentanediol yields as high as 60% in this process. The pentanediol, hydroxymethyl tetrahydrofuran, and methyl tetrahydrofuran formed after hydrogenolysis can be reformed using zeolites to form gasoline-range aromatics or can be hydrotreated to n-pentane and n-hexane for gasoline blending.

Since cellulose and hemicellulose are polymers containing five and six carbon monomers, the hydrocarbons produced from cellulose and hemicellulose will mostly be gasoline range hydrocarbons. But it is also desirable to produce jet and diesel range hydrocarbon fuels from biomass. The chemistry does not favor the production of middle distillates from cellulose and hemicellulose. Fats and lipids are the renewable feedstocks that preferred over cellulosic biomass for the production of renewable jet fuel due to the longer carbon chain lengths found in fats and lipids.

Many research efforts have been made to include cellulosic sources for middle distillates. One example was to make jet fuel from biomass derived levulinic acid via the formation of isobutene. Another example was to use biomass derived oxygenates to produce jet fuel range hydrocarbons via aldol condensation chemistry followed by dehydration and hydrogenation. A third effort was based on co-feeding cellulosic biomass with triglycerides to make jet fuel-range hydrocarbons via two stage biomass catalytic cracking, hydrocracking, and isomerization. None of these proposed processes have proven to be cost competitive with petroleum sourced jet and diesel fuels.

The Renewable Fuels Standard mandates that 21 billion gallons of advanced biofuels will need to be produced by 2022. A part of these advanced biofuels will be fungible transportation fuels such as gasoline, jet fuel, and diesel derived from biomass. Efforts continue on producing such fuels from biomass to meet the mandate and it is perceived that there would be a strong demand for jet and diesel fuels produced economically from biomass.

### BRIEF SUMMARY OF THE DISCLOSURE

The invention more particularly relates to a process for forming C10+ hydrocarbons from biomass wherein biomass is converted to one or more C5 and/or C6 alcohols by hydrolysis of cellulose and/or hemicellulose. The C5 and/or C6 alcohols from the hydrolysis step are catalytically converted into C10 to C12 oxygenates over an alcohol condensation catalyst at catalytically active conditions. The C10 to C12 oxygenates are then hydrotreated to remove oxygen atoms and form saturated C10 to C12 alkane hydrocarbons.

### BRIEF DESCRIPTION OF THE DRAWINGS

A more complete understanding of the present invention and benefits thereof may be acquired by referring to the follow description taken in conjunction with the accompanying drawings in which:

FIG. 1 is a diagram of a system for practicing the present invention;

FIG. 2 is a diagram of an alternative system for practicing the present invention;

FIG. 3 is a diagram showing a first example process reaction pathway of the present invention;

FIG. 4 is a diagram showing a second example process reaction pathway of the present invention;

FIG. 5 is a diagram showing a third example process reaction pathway of the present invention; and

FIG. 6 is a diagram showing a fourth example process reaction pathway of the present invention.

### DETAILED DESCRIPTION

Turning now to the detailed description of the preferred arrangement or arrangements of the present invention, it should be understood that the inventive features and concepts may be manifested in other arrangements and that the scope of the invention is not limited to the embodiments described or illustrated. The scope of the invention is intended only to be limited by the scope of the claims that follow.

In the present invention, a different approach has been taken for the conversion of oxygenates to hydrocarbons. The present invention is related to the conversion of cellulose and hemicellulose into alcohols and polyols such as pentanediol and hydroxymethyl tetrahydrofuran and then converting those alcohols and polyols into jet fuel-range hydrocarbons. The production of alcohols and polyols from biomass may be accomplished with a basic hydrolysis process of cellulose and hemicellulose to sugar alcohols.

There are multiple technologies for the production of sugars and furan oxygenates from cellulosic carbohydrates. A common methodology for the production of sugars and furans from cellulosic carbohydrates involves carbohydrate hydrolysis under acidic conditions. In short, cellulosic materials are put in contact with a solution containing a mineral acid. The concentration of the acid can vary from low concentrations (<1 wt % acid) for mild hydrolysis to high concentrations (1-5 wt % acid) for more intense hydrolysis of the cellulosic carbohydrates. Different schemes have been implemented for processing. In one scheme, steam is directly injected into the biomass acid mixture in a tubular reactor. After a short residence time in the reactor, the resulting slurry and sugar mixture is separated. The acid is neutralized and the sugar solution is cleaned from particle and other impurities. In a different scheme the slurry of cellulosic material and acid solution goes through a series of compression and decompression steps with continuous removal of soluble carbohydrate hydrolysis products. In another scheme, near critical water is injected to a biomass slurry for carbohydrate hydrolysis at high pressures. Biochemical routes where enzymes have been applied for carbohydrate depolymerization into monomeric sugars have also been disclosed.

Independent of the scheme used to obtain cellulosic sugars, sugars can be hydroprocessed into sugar alcohols, polyols, monoalcohols and hydrocarbons using high pressure hydrogen and a metal base—solid catalyst. Sugar hydrogenolysis has been practiced with different metal catalysts, but Ru—based catalysts are usually most effective for the application. After hydrogenolysis a polyol feed is obtained that can be further processed into larger hydrocarbons using the methods disclosed in this invention.

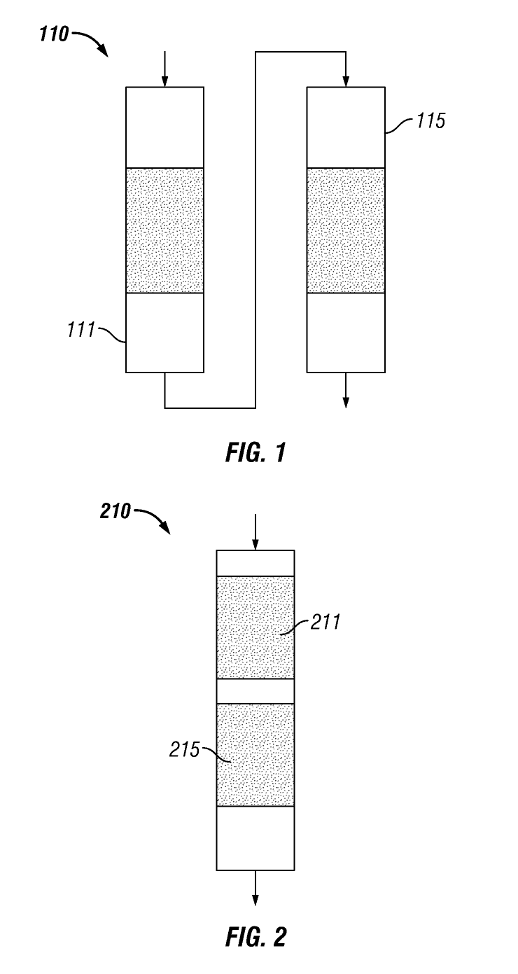

As shown in FIG. 1, the process 110 is performed in a fairly straight forward arrangement where an alcohol feedstock from a biomass hydrolysis system (not shown) is delivered to reactor 111 which includes heterogeneous alcohol condensation catalysts to convert the C5 and C6 alcohols to C10+ oxygenates. The C10+ oxygenates are then passed to hydrotreater 115 where the oxygenates are saturated and hydrodeoxygenated to form C10+ alkanes. The advantage of this arrangement is that both reactions can occur at different reaction conditions, if warranted. Other practical systems for performing the invention may be created. For example, the process may also be performed in a two stage reactor shown in FIG. 2 where the process 210 includes a first bed 211 for converting C5 and C6 alcohols to C10+ oxygenates and a second bed for hydrotreating the C10+ oxygenates to form C10+ alkanes. Both reactions will take place in the temperature range of 250-350° C. and a pressure range of 400-1400 psig.

Suitable catalysts for alcohol condensation have both base sites and metal sites. In this regard, basic oxides with metal promoters are the best for this application. Metal promoters include metals from groups VI to XII of the periodic table. Basic oxides include oxides from mainly group II, but any oxide with base properties can be used for this application. Some of the common catalysts include: Mg—Al based bi-functional catalysts, Cu-based catalysts, supported noble metals, and alkali or alkaline earth metal hydroxides.

In the first reactor 111 or first reactor catalyst bed 211, the chemistry being performed is a series of reactions over the catalyst to first form aldehydes and then by condensation form dimers of these molecules. The broad process is known as a Guerbet chemistry. The Guerbet reaction is a condensation reaction of primary alcohols to yield a β-alkylated dimeric alcohols with loss of water. The reaction involves two main steps: 1) alcohol dehydrogenation to aldehydes, and 2) subsequent aldol condensation reaction of the aldehydes. Both these steps take place in-situ and are not stand-alone steps.

Guerbet chemistry has been widely studied for making higher alcohols or oxygenates from C1-C3 alcohols. To the extent that others have studied Guerbet chemistry to form higher oxygenates, none of them have extended the chemistry further such as to make hydrocarbon fuels from the oxygenates as disclosed in this invention. Others have focused on the production of synthetic detergents, cosmetics, and lubricants using Guerbet chemistry.

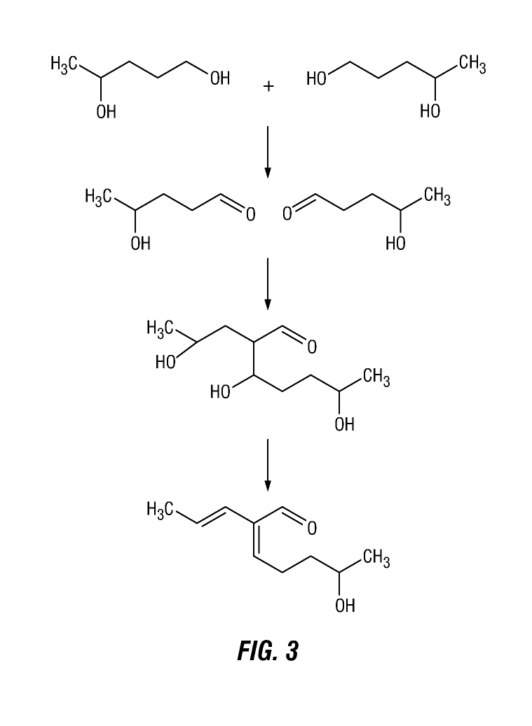

Applying Guerbet chemistry to the present invention, a C5 or C6 alcohol created through a biomass hydrolysis process, such as pentanediol or hydroxymethyl tetrahydrofuran may be converted to C10 oxygenates by means of a number of different in-situ pathways. For example, pentanediol, by itself, may be dimerized to form one of several C10 oxygenates through three different pathways where the first is shown in FIG. 3. The first pathway is a self-aldol condensation process involving the terminal alcohol group or terminal hydroxyl group of the pentanediol molecule. This hydroxyl group dehydrogenated to carbonyl group at the terminal end forming an aldehyde or an aldol. Two such similar aldols are dimerized to form carbon-carbon bonds briefly forming a C10 triol with a carbonyl group on one branch. Dehydration of the C10 triol can give rise to unsaturated C10 aldol or C10 oxygenate. Dehydration products can also undergo C—C bond coupling reaction resulting in heavier oxygenates/hydrocarbons.

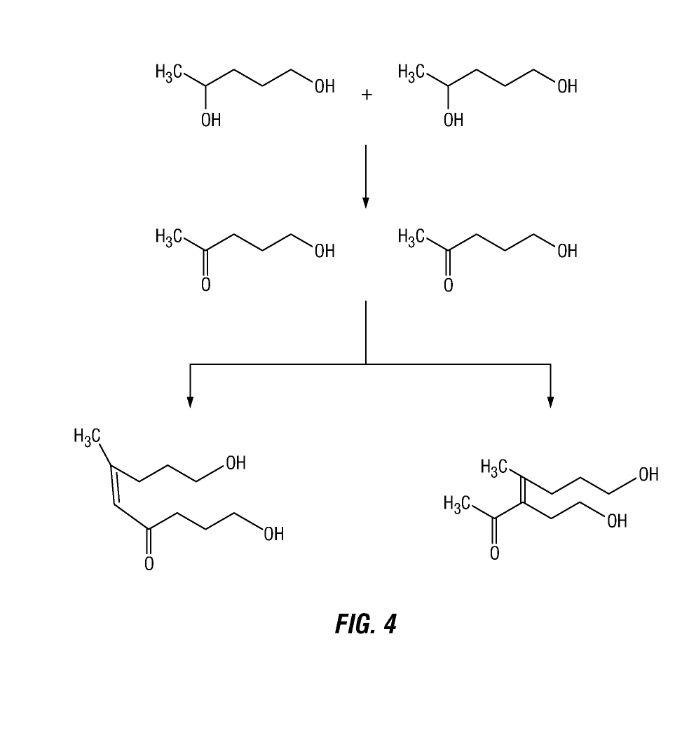

The second pathway is similar to the first but involves the branched alcohol or hydroxyl group on the pentanediol as shown in FIG. 4. The hydroxyl group attached to the beta carbon is stripped of its hydrogen resulting in a double bonded oxygen or carbonyl group at the beta position of what is now a ketone or an aldol. Two such similar aldols are again dimerized, but can bond in at least two arrangements as shown in FIG. 4. The resulting molecule is a C10 aldol. In both paths, water can be formed concurrently with the forming of the C10 aldol or oxygenate molecule.

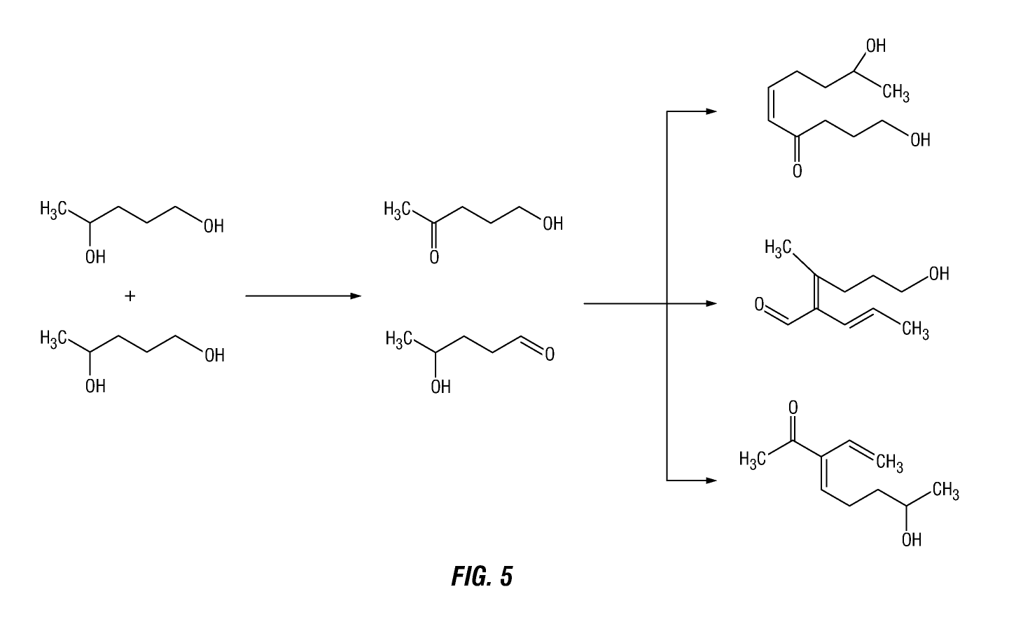

The third pathway for pentanediol is cross-aldol condensation where two different aldols come together for dimerization. The two different aldols are the described in the first two pathways. The two aldols are able to combine on the first catalyst to form a C10 molecule, where three examples as shown in FIG. 5. After hydrotreating, and the oxygenates have been converted to alkanes, the alkane molecules 5 that are formed via this third pathway after hydrotreating are decane, 4,5-dimethyloctane and 3-ethyloctane.

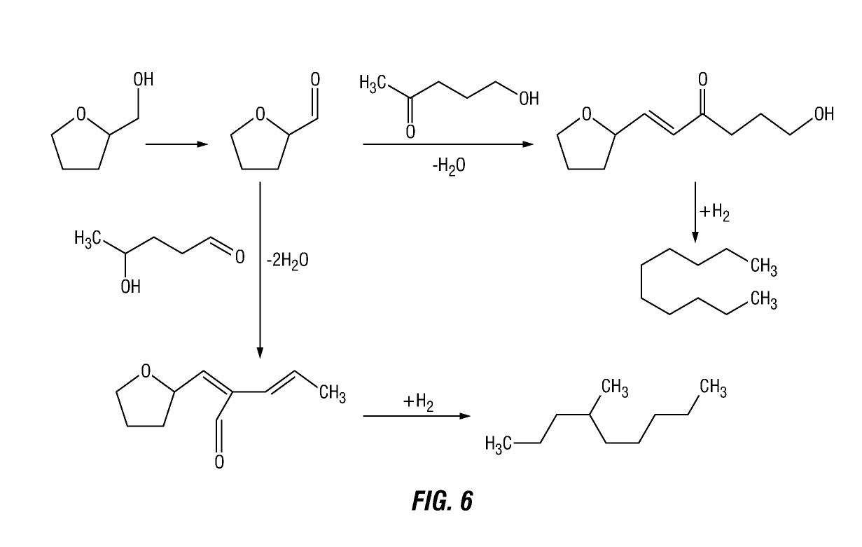

The inventive process includes other similar reactions of C5 and C6 alcohols to larger alkanes, as the sugar hydrogenolysis step produces a great distribution of polyols and alcohols. Another example describe here is the reaction of pentanediol with hydroxymethyl tetrahydrofuran. This is shown in FIG. 6. Each is subjected to catalyst dehydrogenation step of a respective hydroxyl group forming hydroxymethyl tetrahydrofuran aldehyde and pentane aldol. The aldehyde and aldol combine to form a C10 oxygenate molecule that is generally linear or a branched C10 molecule.

The products of the aldol condensation reaction are then hydrogenated and hydrodeoxygenated in a second reactor to produce a variety of C10+ alkanes. These C10+ alkanes are derived from biomass through a hydrolysis process, then through a Guerbet condensation process and then then through hydrotreating. The alcohols from the hydrogenolysis process may be selected for the aldol condensation process by fractionation or other selection process. The hydrocarbons produced at the end of the process are in jet fuel-range distillation range, but may also be used in diesel fuel. The hydrocarbons are linear, branched, naphthenic, and aromatic, as dimerization products as explained above tend to recombine and undergo other secondary reactions. The hydrocarbon products are likely to be blended with petroleum derived jet and/or diesel. All of the reactions occur on heterogeneous catalysts at elevated temperature and pressure.

Hydrotreating is a mature technology used in crude oil refining for the elimination of heteroatoms (sulfur, nitrogen and oxygen) from hydrocarbons using hydrogen at moderate-to-high temperatures and pressures. Hydrotreating is conventionally carried out in packed bed reactors with supported sulfided metal catalysts. Typical hydrotreating catalysts are cobalt/molybdenum sulfides on alumina supports. Other hydrotreating catalysts include sulfided nickel-molybdenum or cobalt-molybdenum on alumina or zeolitic supports. Supported noble metals have also been used for hydrotreating applications. The typical hydrotreating reaction is carried out in the temperature range of 200-350° C. and in the pressure range of 800-1400 psig.

It could be possible to convert cellulosic alcohols to jet fuel-range hydrocarbons in single pass rather than having multiple steps. In this scheme a stacked bed reactor as shown in FIG. 2 is used. The top bed contains a catalyst bed with a base solid catalyst promoted with a metal as previously explained. Hydrogen is injected on the bottom bed for hydrotreating. Hydrotreating in this case is carried out with a sulfur free metal-based catalyst.

While the examples have focused on C10 alkanes, C10, C11 and C12 alkanes are anticipated to be produced by the dimerization of C5 and C6 alcohols where two C5 alcohols will make a C10, a C5 and a C6 will make a C11 and two C6 alcohols will make a C12 alkanes. Other hydrocarbon types are also formed by further transformations of oxygenate products from alcohol condensation reactions.

In closing, it should be noted that the discussion of any reference is not an admission that it is prior art to the present invention, especially any reference that may have a publication date after the priority date of this application. At the same time, each and every claim below is hereby incorporated into this detailed description or specification as additional embodiments of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims while the description, abstract and drawings are not to be used to limit the scope of the invention. The invention is specifically intended to be as broad as the claims below and their equivalents.

### REFERENCES

All of the references cited herein are expressly incorporated by reference. The discussion of any reference is not an admission that it is prior art to the present invention, especially any reference that may have a publication data after the priority date of this application. Incorporated references are listed again here for convenience:
1. Bond, J. Q., Alonso, D. M., Wang, D., West, R. M. and Dumesic, J. A, “Integrated Catalytic Conversion of yValerolactone to Liquid Alkenes for Transportation Fuels” Science 327 (2010) 1110-1114.
2. Dumesic, 1. A, Huber, G. W., Chheda, J. N. and Barrett, C. 1., “Stable, Aqueous-Phase, Basic Catalysts and Reactions Catalyzed Thereby”, US2008/0058563 AI, Wisconsin Alumni Research Foundation, 2008.
3. Dumesic, 1. A and Roman-Leshkov, Y, “Production of Liquid Alkanes in the Jet Fuel Range (C8-C15) from Biomass Derived Carbohydrates”, US200910124839 AI, Wisconsin Alumni Research Foundation, 2009.
4. Dumesic, 1. A., Simonelli, D. A and Kunkes, E. L., “Single-Reactor Process for Producing Liquid-Phase Organic Compounds from Biomass”, WO 20091129019 A1, Wisconsin Alumni Research Foundation, 2009.
5. O'Connor, P. and Yanik, S., “Producing Fuel and Specialty Chemicals from Biomass Containing Triglycerides and Cellulose”, WO2010/002886 AI, K1OR Inc., 2010.
6. O'Lenick, A J. J., “Guerbet chemistry” J. Surfactants and Detergents 4 (2001) 311-315.
7. Hsiao, T. C. and Lin, S. D., “Methanol to Long-chain Oxygenates Over Mg1A1 Mixed Oxides Supported Cu Catalysts” Catal. Lett. 119 (2007) 72-78.
8. Mueller, G., Gutsche, B., Jeromin, L., Steinbemer, u., Sedelies, R., Bohlander, R., Ridinger, R., Springer, D., Buettgen, F. and Bartschik, F., “Process for the production of Guerbet alcohols” U.S. Pat. No. 5,777,183 Henkel KommanditgeseUschaft auf Aktien (1998).
9. Cripe, T., Conner, D., Vinson, P., Burckett, L., James, C. and Willman, 1., “Polyoxyalkylene surfactants” U.S. Pat. No. 6,093,856 Procter and Gamble Co. (2000).
10. Cripe, T., Conner, D., Vinson, P., Burckett, L., James, C. and Willman, 1., “Mid-chain branched alkyl sulfate surfactants” U.S. Pat. No. 6,060,443 Procter and Gamble Co. (2000).
11. O'Lenick, A J. J., “Guerbet based sorbitan esters” U.S. Pat. No. 6,013,813 Hansotech Inc. (2000).
12. O'Lenick, A 1. 1., “Alkoxylated Guerbet alcohols and esters as metal working lubricants” U.S. Pat. 4,731,190 Alkaril Chemicals Inc. (1984).
13. Vinson, P., Foley, P., Cripe, T. and Connor, D., “Liquid cleaning compositions containing selected mid-chain branched surfactants” U.S. Pat. No. 6,046,152 Procter and Gamble Co. (2000).
14. Warner, J., “Alkoxylated primary alcohol surfactants providing enhanced efficacy and/or rainfastness to glyphosate formulations” U.S. Pat. No. 5,663,117 Monsanto Co. (1997).
15. Carlini, C., Marchionna, M., Noviello, M., Raspolli-Galletti, A M., Sbrana, G., Basile, F. and Vaccari, A, “Guerbet condensation of methanol with n-propanol to isobutyl alcohol over heterogeneous bifunctional catalysts based on MgA1 mixed oxides partially substituted by different metal components” J. Mol. Catal. A: Chern. 232 (2005) 13-20.
16. Carlini, C., Di Girolamo, M., Marchionna, M., Noviello, M., Raspolli-Galletti, A M. and Sbrana, G., “Selective synthesis of isobutanol by means of the Guerbet reaction Part 1. Methanolln-propanol condensation by using copper based catalytic systems” J. Mol. Catal. A: Chern. 184 (2002) 273-280.
17. Matsuda, M. and Horio, M., “Process for preparation of Guerbet alcohols” U.S. Pat. No. 4,518,810 Kao Corporation (1985).
18. Clark, R. T., “Vapor-phase conversion of methanol and ethanol to higher linear primary alcohols by heterogeneous catalysis” U.S. Pat. No. 3,972,952 Celanese Corporation (1976).
19. Griffm, T. P., “Reactive Separation to Upgrade Bioprocess Intermediates to Higher Value Liquid Fuels or Chemicals”, US2009/0182064 AI, Pennsylvania Sustainable Technologies, LLC, 2009.

1. A process for forming C10+ hydrocarbons from biomass comprising:
a) converting biomass to one or more C5 and/or C6 alcohols by hydrolysis of cellulose and/or hemicellulose;
b) catalytically converting the C5 and/or C6 alcohols from step a) into C10 to C12 oxygenates over an alcohol condensation catalyst at catalytically active conditions; and
c) hydrotreating the C10 to C12 oxygenates to remove oxygen atoms and form saturated C10 to C12 alkane hydrocarbons.

2. The process according to claim 1, wherein the alcohol condensation catalyst is a base catalyst.

3. The process according to claim 1, wherein the condensation catalyst is a hetero catalyst.

4. The process according to claim 1, wherein one of the alcohols is a pentanediol.

5. The process according to claim 1, wherein one of the alcohols is a hydroxymethyl tetrahydrofuran.

6. The process according to claim 1, wherein step b) first comprises converting alcohols to aldols and further comprises dimerizing two aldols to form the C10 to C12 oxygenates.

7. The process according to claim 6, wherein step b) further produces water with the oxygenates.

8. The process according to claim 1, wherein the produced alkanes are both branched and straight chain alkanes.
